(19) United States
(12) Patent Application Publication
(10) Pub. No.: US 2006/0173345 A1
(43) Pub. Date: Aug. 3, 2006

(54) Strip transducer for large aperture ultrasonics

(76) Inventor: James K. Bullis, Sunnyvale, CA (US)

Correspondence Address: James K. Bullis, 1155 Pimento Ave., Sunnyvale, CA, 94087, US

(21) Appl. No.: 11/043,631

(22) Filed: Jan. 25, 2005

(51) Int. Cl. A61B 8/14

(52) U.S. Cl. 600/459

(57) ABSTRACT

The present invention provides a critical breakthrough in large aperture ultrasonic transducer technology, enabling a variety of large ultrasonic devices that emulate optical lens functions. The present invention is a transducer assembly and method of production. The assembly includes metalized transducer material and additional materials in a design that enables both effective ultrasonic functionality and efficient manufacturing. Each assembly includes a strip of the transducer material, usually two strips of foundation material, and at least one strip of supporting material. The manufacturing process utilizes structural properties of the foundation material and supporting material in manufacturing tooling to enable efficient fabrication of transducer assemblies from a flat card of transducer material. Foundation material and the supporting material remain as part of the assembly. Because these materials are also selected for ultrasonic properties, they support effective ultrasonic operation of that assembly.

This patent document contains material that is subject to copyright protection. Facsimile reproduction is allowed of the patent document or the patent disclosure as it appears in the Patent and Trademark Office patent file or records as allowed by US patent law, but otherwise all copyright rights are reserved.

### CROSS REFERENCE TO RELATED APPLICATIONS

This invention improves on devices disclosed in co-pending application Ser. No. 10/667,547 filed Sep. 22, 2003 by the present inventor.

### BACKGROUND OF THE INVENTION

1. Field of the Invention

This present invention relates to ultrasonic technology, including medical imaging technology, medical treatment technology, and industrial inspection technology. Other fields where ultrasonic waves are involved are also included.

2. Description of the Prior Art

Important developments have taken place in ultrasonic technology, but such progress has been limited by limited availability of large aperture ultrasonic transducers, especially where independent operation of transducer elements are of interest, as in transducer arrays. As a result, the field of ultrasound is often based on adaptations of radar and sonar approaches and the high resolution of optical systems remains elusive. In order to significantly improve resolution of ultrasonic systems the fundamental physics that limits resolution must be addressed, where short wavelength and large physical dimensions of transducer arrays enable reduce resolution cell size in lateral directions. Reduced resolution cell size corresponds to higher resolution of a system. Optical devices commonly achieve very low ratios of wavelength to aperture size.

Interesting possibilities in ultrasonics exist for a more powerful approach, where two lateral dimensions are resolved by aperture effects and time resolution of the range dimension is used primarily for refining depth of field effects. This is in contrast to radar and sonar based approaches where only one dimension is resolved by aperture effects and the other by time resolution of the range dimension. Such approaches are seriously vulnerable to clutter that is poorly suppressed in one lateral dimension. With the more powerful approach where more resolution power is utilized, it appears possible to better contend with the difficulties of volumetric imaging in body tissue as needed for good medical imaging.

Particular interest is in developing of ultrasonic methods for imaging of the female breast to screen for breast cancer, where up to now the quality of ultrasound technology has not been adequate.

There are other considerations in developing of better ultrasonic systems, but a key limitation is the difficulty of producing large aperture transducer arrays, where aperture is needed in two dimensions. A previous patent application disclosing a spot focusing system, Ser. No. 10/667,547 Bullis Sep. 22, 2003, also included a solution to this aperture size limitation problem. This was a strip transducer concept that enabled large aperture transducers having general shape. This method there discussed is satisfactory for lower frequency operation, but falls short where transducer strips suitable for operation at the upper range of frequencies of interest, the main difficulty being in achieving reasonable yield from a production process.

Beyond the above mentioned spot focused system, an even more general benefit is envisioned based on a wavefield transformer device, Ser. No. 10/060,591 Bullis Jan. 30, 2002, where a method of arranging emulations of general optical devices was discussed. Full utilization of the wavefield transformer concept requires producible, large aperture, transducer arrays that can operate effectively at high frequencies.

### SUMMARY OF THE INVENTION

The present invention provides a critical breakthrough in large aperture ultrasonic transducer technology. This would appear to make possible a great expansion in ultrasonic devices. New possibilities include two dimensional aperture systems that operate more comparably to optical devices while retaining the special qualities of time based operation. Beyond this, more general utility is expected since a variety of large ultrasonic devices can now be formed that emulate optical lens functions.

The present invention is a transducer assembly and method of production. It is a novel combination of metalized transducer material and additional materials that enables both effective ultrasonic finctionality and efficient manufacturing. Each assembly includes a strip of the metalized transducer material, usually two strips of foundation material, and at least one strip of supporting material. From a card of metalized transducer the intended product from each such card is a number of such transducer assemblies. The manufacturing process utilizes structural properties of the foundation and supporting material in manufacturing tooling. However, foundation material and the supporting material remain as part of the product, having a second purpose where ultrasonic operation of the transducer assembly utilizes ultrasonic properties of these materials.

To begin the manufacturing process, cards of foundation material are attached on both sides of the card of transducer material to form a card sandwich. A first set of grooves is then cut in the card sandwich. The supporting material is then used to fill these grooves. The supporting material is a combination of liquid and solid parts that cure in place without significant dimensional change. After the supporting material has cured in place, the manufacturing process involves cutting of a second set of interleaved, parallel grooves that then define the very thin strips of transducer material. Typically two strips of foundation material and at least one remnant strip of supporting material remain as a part of the transducer assembly. Trimming of foundation material and supporting material are precisely done to enable the intended ultrasonic operation. Cutting then separates and completes formation of the assemblies and electrical connections are also arranged.

The foundation material and the supporting material must have properties that are suitable for providing structural support to the transducer material so that it can be cut in a very thin strip. They also serve to make the assembly rugged and convenient to handle such that a reasonable manufacturing yield results. The foundation material then serves as a pressure release surface to enable ultrasonic displacement of the transducer material strip and to cause directional blockage of wave propagation. The supporting material serves to support directional wave propagation through itself.

The transducer assemblies may be operable as a single strip transducer element or the strips may be segmented such that each assembly is operable as a plurality of transducer elements.

### BRIEF DESCRIPTION OF THE DRAWINGS

FIG. 1 shows two views of a strip transducer assembly, where part (a) is a view showing an edge of a strip of transducer material and part (b) is a view from the opposite side.

FIG. 2 shows two views of the transducer material strip.

FIG. 3 shows two views of a supporting material strip.

FIG. 4 shows two views of foundation material formed to provide a flange and to support the lower side of the transducer material strip after cutting operations.

FIG. 5 shows two views of foundation material that remains on the upper side of the transducer material strip after cutting operations.

FIG. 6 shows a sandwich of a card of transducer material and an upper card and a lower card of foundation material.

FIG. 7 describes a sequence of cutting and filling operations that produce the transducer assembly.

FIG. 8 shows a cross section through the center of the strip transducer.

FIG. 9 shows a cross section through the center of the strip transducer as mounted on a backing block.

FIG. 10 shows a cross section through the center of the strip transducer as mounted on a backing block, except that the supporting material is a continuation of the transducer material that is modified.

FIG. 11 illustrates the manufacturing sequence to include a second strip of supporting material on the opposite side from the first strip of supporting material.

FIG. 12 illustrates the manufacturing sequence to produce a segmented strip transducer.

FIG. 13 shows transducer material and electrical connections for a segmented strip transducer.

### DETAILED DESCRIPTION OF THE PREFERRED EMBODIMENTS

This invention enables a new concept in ultrasonic transducer arrays, where large arrays are formed using strip transducers. It is a functional product design concept for strip transducer assemblies suitable for forming large aperture transducer arrays and a practical manufacturing method for such strip transducers. The strip transducers needed may be either straight-line strips or curved strips. They may be continuous strips that operate as a single transducer that is an element in an array of transducers or they may be segmented strips that operate as a sub-array of transducers, where sub-arrays form the full transducer array system.

The full transducer arrays are generally used with electronic systems that scan the focal direction or the focal distance. However, the arrays may be fixed in shape to provide propagating wave related effects that are simply related to the three dimensional geometry of the array surface with parallel signal operation where signals are all in phase. Transducer systems will typically use many strip transducers assemblies in the large transducer system. The geometric relationships of such assemblies, and elements of the sub-arrays where such are involved, require significant relative position control accuracy. This requires a unified mounting system such as a backing block. The backing block may also be useful for absorbing ultrasonic waves that are not desirable.

Transducers operate in relation to waves, either to transmit or receive such waves, and they convert between electrical signals and wave signals. Electrical operations are arranged as appropriate. A common issue with transducers is the phase shift that they cause over the frequency band, where this depends on a variety of details in the construction of the transducer assembly. Where necessary, signals can be used that compensate for such phase shift.

The present invention is a design that has dual utility, both as an operating transducer device that satisfies the requirements mentioned above and as an assembly having features that enable efficient manufacturing of that assembly. The approach and its variations that are discussed here serve to illustrate the general concept of the approach that is the present invention.

A transducer assembly includes a thin strip of transducer material that is relatively long compared to its cross sectional dimensions. The length of the strip is sufficient to effectively clamp motions along its length. The cross section is rectangular. This thin strip has conductive surfaces that are placed on two opposite surfaces. These act to accumulate charge in relation to electric field effects that arise in the transducer material. The other two surfaces serve as the intended ultrasonic operating surfaces, one of which is facing into a medium for ultrasonic operation and the other faces into the backing block. In fact, the surfaces move to compensate such that the actual volume of the block does not significantly change, so width changes in one of the cross sectional dimensions are about equal to width changes in the other cross sectional direction. Given this fact, it makes little difference which pair of parallel sides has the conductive surfaces attached. The sides used in the illustrated embodiments are chosen for manufacturing reasons, where the available raw material is a transducer card with flat surfaces that are metalized. Whichever pair of parallel sides includes the intended ultrasonic projecting side, the orthogonal pair of parallel sides must be allowed to move. Mounting that would clamp so as to prevent the orthogonal side motion would reduce ultrasonic effectiveness of the transducer assembly.

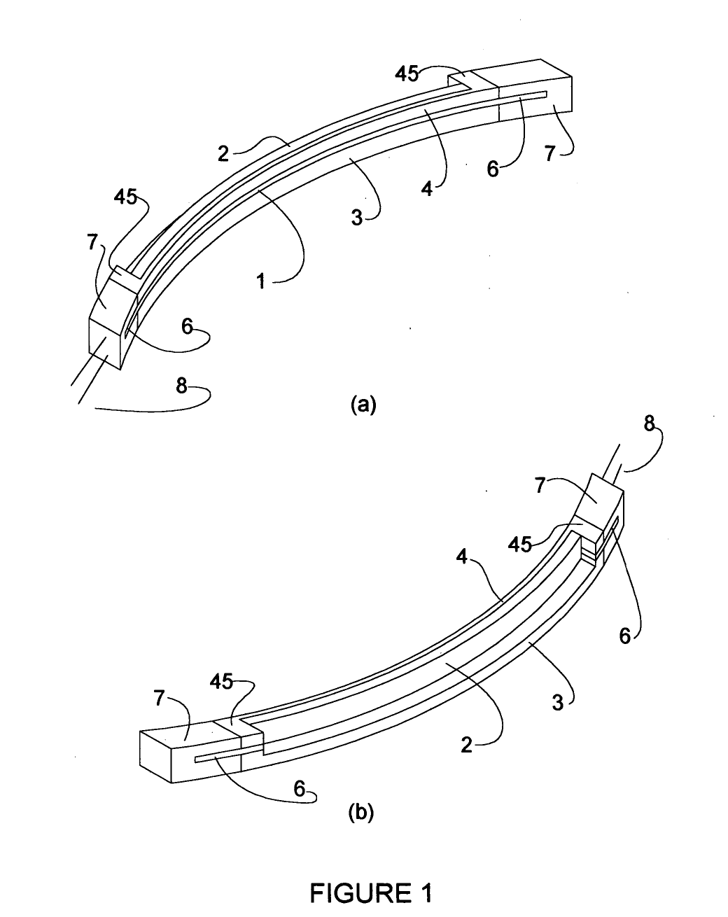

The above thinking is the basis of the configuration that is shown in FIG. 1. The active ultrasonic surface of transducer material 1 is visible from the viewing perspective in part (a) of this illustration. Here the transducer material is at the surface, except for a thin waterproof coating that is not shown. A supporting material strip 2 is visible in both part (a) and (b) of this figure. A lower layer of foundation material 3 is also shown, with a flange that is mostly visible in part (b). An upper layer of foundation material 4 is also shown. The supporting material is a hard epoxy, part of which was hardened in place and part of which was inserted as a solid. The foundation material is called a foam material that is very low density, being a composite of epoxy and a powder material that is actually very small, hollow glass spheres. Tabs of transducer material 6 extend outward at the strip ends with support to the tabs provided by enclosures 7, where the enclosures are of enclosure material that is a foam like material that is much like the foundation material. Wires 8 protrude from the end enclosure material. A transition region 45 supports the ultrasonically active part of the strip transducer part 4 from the end enclosure 7. This is designed to enable hold down clamping during manufacturing. It could be said that all materials support the transducer strip, but terminology here distinguishes between “supporting material”, “foundation material”, and “end enclosure material”, where there are key differences that are important features of this invention.

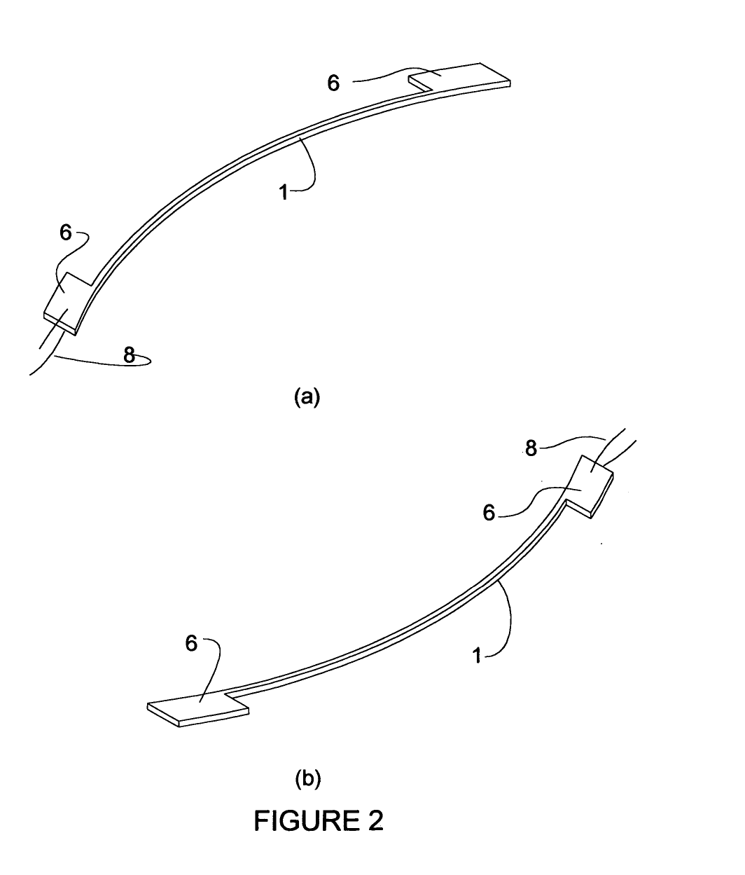

FIG. 2 shows corresponding views of the transducer material part of the transducer assembly. The thin section is formed to operate at a high ultrasonic frequency. In the immediate application under development this thin section is too small to be visible if shown at true scale. Consequently, this illustration, as well as the other corresponding views, are greatly distorted in scale, where length is roughly 2 times actual and cross section dimensions are roughly 10 times actual. The length of the active strip transducer part prevents significant displacement along the length dimension. The end tabs 6 are of relatively large such that very little displacement occurs. Slight thickness displacement occurs, but this is not coupled to the medium of propagation. Both sides of the tabs 6 are metalized with electroless nickel against the piezo-electric transducer material and electroplated copper on top of the nickel surface. Metalization continues from the tab to the thin strip 1 such that a continuous conductive path is present over the full length of the thin strip 1. Wires 8 are soldered to both sides of the tabs 6.

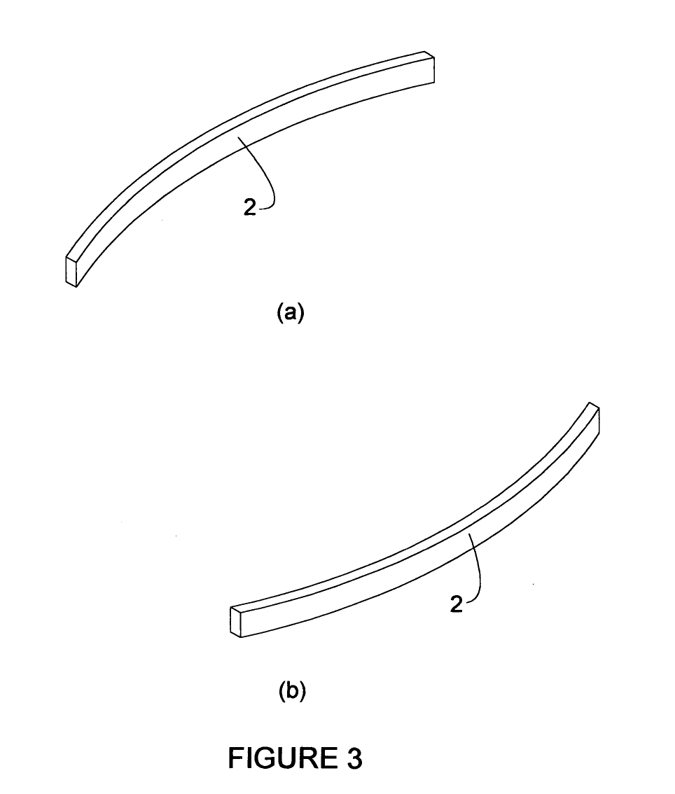

FIG. 3 shows corresponding views of the supporting material strip 2 that is attached to the back side of the transducer material strip 1 from previous figures. It adds significant structural strength to the assembly and enables ultrasonic waves to flow through it.

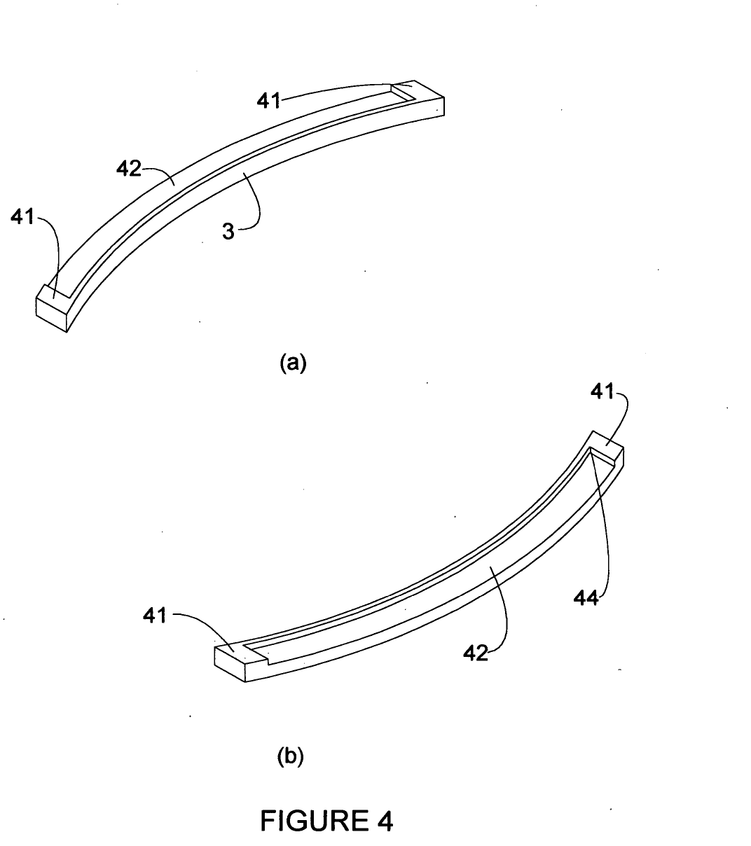

FIG. 4 shows corresponding views of the lower foundation strip 3 that is formed into a flange 42. This flange is designed to enable positioning of the assembly in a transducer array system. The widened parts 41 support the tabs 6 of the transducer material shown in the previous figure. Comers 44 shown as square are actually of radius that is determined by the radius of a cutter, which is to be described later in this disclosure. This lower foundation strip impedes flow of ultrasonic waves that would tend to go through it.

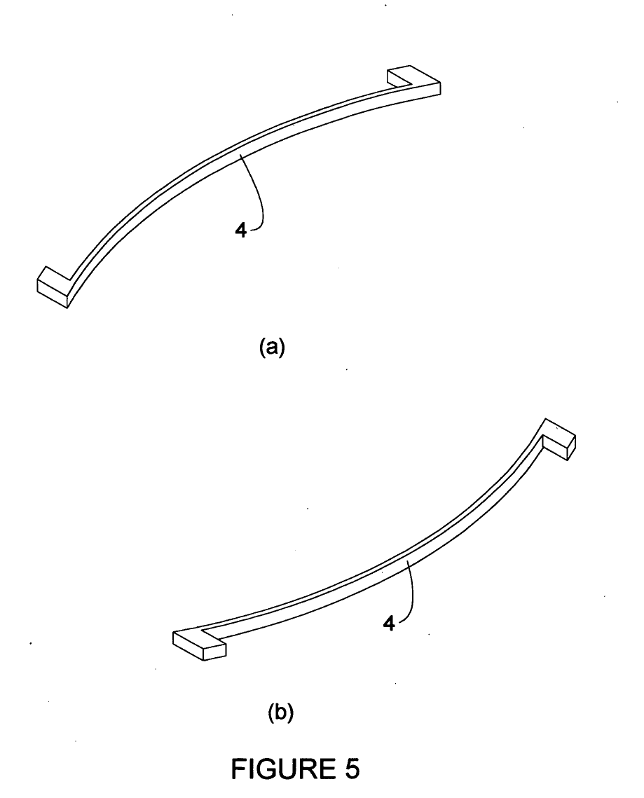

FIG. 5 shows corresponding views of the upper foundation strip (not exactly an appropriate name). It functions in the same way as the lower foundation strip, except it does not have a flange. Note that the final thickness of both foundation strips is designed to be very thin to enable close spacing of adjacent transducer assemblies in an array system.

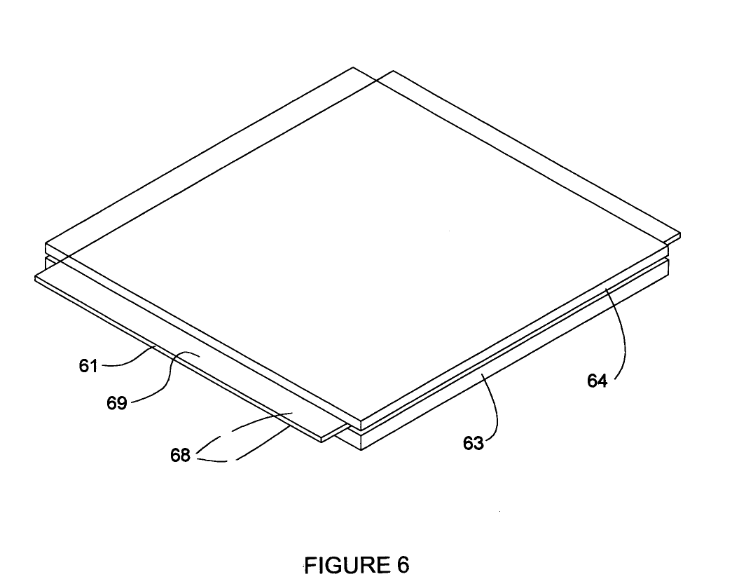

Discussion of the method of manufacturing that is key to this invention begins with FIG. 6, which illustrates a preliminary assembly that is the basis of a process to produce many strip transducer assemblies. The card of piezo-electric material 61 is shown in the middle, with metalized surfaces 69 exposed at the edges. On the bottom and the top are cards of low density foam 63, 64, respectively. Attachment of wires is indicated by the attachment of a single pair of such wires 68, as shown. This card sandwich is to be cut into many strips, so that many pairs of wires are required.

In current practice, the piezo-electric card is 2 inches by 3 inches and 0.008 inch thick, as purchased from Morgan Electro Ceramics. They call it “plate.” It comes with a very thin nickel conductive material plated on both the flat surfaces. This is then electroplated with copper to a thickness of approximately 0.0005 inches.

The foam cards are 0.037 inches and 0.014 inches respectively. They are made by mixing epoxy from Abotron, Inc, Resin 50-24, 20 parts with Hardener 8501-4, 23 parts, with glass spheres from 3M Company, Scotchlite H50/10,000 EPX, 71 parts (all parts by weight). The process involves mixing, vacuuming to remove air, casting on a flat surface, rolling under a teflon sheet, and curing as instructed, with the high temperature phase reduced to 150 degrees C. The cards are then ground using a diamond mandrel over a flat table, held down by vacuum and water flooded for cooling and lubrication of the cutter/grinder mandrel. The lower foundation card is ground to the final thickness of the ultimate lower foundation strip part of the transducer assembly.

In most recent production, the top card is left thicker than its final thickness in the final transducer assembly as a measure to increase rigidity during cutting. The actual top card used now is 0.037, since this is a thickness that was available. This entails a trimming operation to get to the 0.014 thickness that is intended in the final assembly.

The total thickness of the sandwich relates to clamping rail devices that are used in the cutting operation. They hold down the edges of the sandwich. The part of the sandwich that is under the clamping rail devices can not be cut without removing the clamp, so this is done after the center cutting of all cuts to be done on the sandwich. The end configuration 44 shown in FIG. 4 allows for this fact, as does the end configuration 45 in FIG. 1. Part of the tab 6 shown in FIG. 2 is also under the clamping rail, and it is also arranged to allow for this.

The sandwich so described is formed by bonding the cards together with the same epoxy formulation described above, except without the glass spheres and except keeping the final cure temperature at 100 degrees C.

It is optional whether to attach wire pairs that are required for each strip transducer assembly on the exposed flat surfaces 69 indicated in FIG. 6 prior to assembly of the card sandwich, prior to the cutting operations that are to be done, or after the strips are separated from each other as the final strip assemblies. Each way has disadvantages. Solder flowing properties cause unwanted thickness of the center card of the sandwich. Solder cutting causes difficulties where the diamond cutters get clogged by the soft solder metal and immediately break. Wires get in the way of clamping devices. Otherwise, cleaning, fluxing, and soldering at later stages are difficult and inefficient processes to be done. A tooling refinement is necessary if wires are to be attached in advance of cutting.

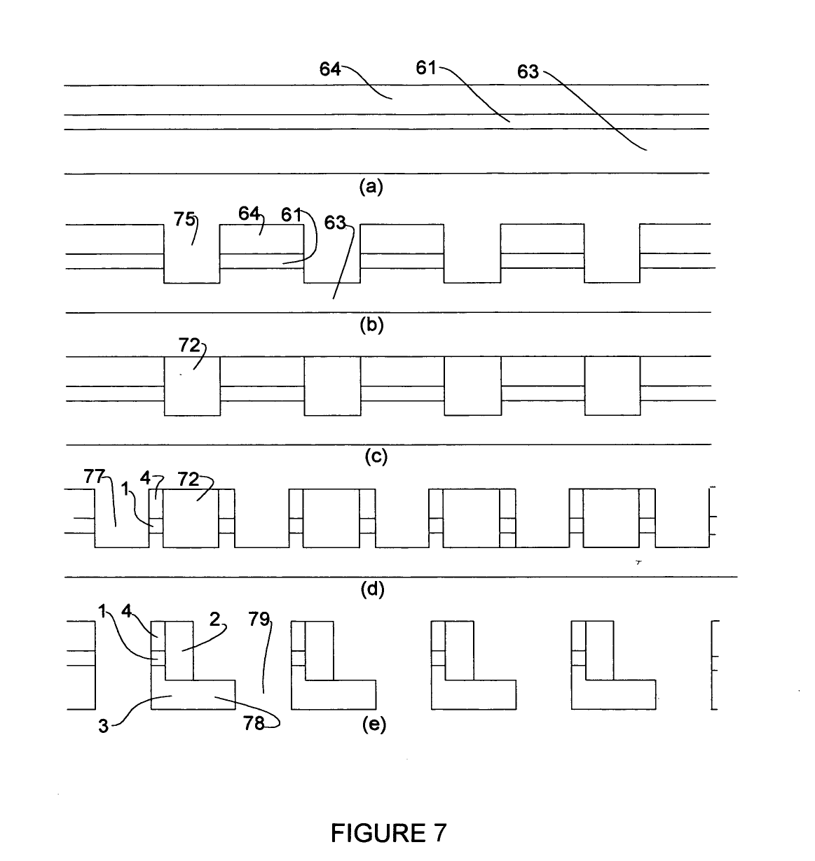

The cutting operations are discussed in relation to FIG. 7, but first a general description of the cutting method is needed. The transducer material is a hard, abrasive, and fragile ceramic. This led to use of a diamond mandrel that operates like an end mill device, used in a computer controlled milling machine that is adapted for this operation. The mandrel needs to be as small in diameter as possible since its size determines how much material is wasted. A 0.040 inch diameter, diamond coated, tool, Starlite part number 115,010, has been used successfully in a spindle running at about 10,000 rpm The copper plating is a soft metal that tends to clog the cutter if too thick, as does the solder if it is present. Clogging tendencies cause peeling which is a seriously strong delaminating effect, though the plating of 0.0005 inches works if it is encased in upporting material. Lower spindle speeds were originally used but abandoned. Now, cutters continue to break, but only occasionally. The manufacturing system is such that cutters can be replaced and the computer controlled cutting system can be indexed to return to the same relative position, this being critical to controlling the final dimensions of the strip transducer components.

The manufacturing method must efficiently produce a significant number of transducer assemblies from each card sandwich. The process involves making each type of cut repeatedly over the surface of the card sandwich. A next type of cut is then done at a precisely controlled position relative to the previous cut. After multiple types of cuts are completed, each being done repeatedly over the card sandwich extent, the significant number of transducers assemblies exists. This is all consistent with the software that was written to control the milling machine and the way supporting material is utilized to enable cutting without fracturing or delaminating the card sandwich materials.

FIG. 7 is useful for discussion of this method of manufacturing. Part (a) shows a cross sectional view through a card sandwich where the transducer material layer 61 is sandwiched between a lower card of foundation material 63 and an upper card of foundation material 64. Part (b) shows the result of the diamond cutter making a first cut to make a groove 75 across the sandwich, cutting through the top layer of foundation material 64, the transducer material layer 61 and partly through the lower layer of foundation material 63.

A sequence of such first cuts are also shown that continue repeatedly over the extent of the card sandwich.

Part (c) then shows the result of filling the grooves with supporting material 72. This supporting material is an epoxy material from Abotron, Inc. that is based on a mix of resin 50-6, 100 parts and hardener 50-12, 14 parts, both parts by weight. The actual filling is made by first casting a slab of this mixture and curing as specified, utilizing the option to cure at 100 degrees C. Then the slab is cut into strips that fit down into the grooves that were cut in the sandwich. They are cut to fill the grooves as completely as possible without cause distortion of the walls of the grooves. The same epoxy is then used to bond these strips in place, thus forming continuous filing material that causes little distortion of the grooves while curing. Curing is done without removing the sandwich from the cutting apparatus table. This method was arrived at after a serious effort to find a clear epoxy that did not change volume on curing. This approach reduces the volume change by about 20 times relative to that where epoxy is simply poured into the grooves. If the same epoxy material is cured and ground into small particles and mixed with that same uncured material, a similar effect can be achieved, though the cut strips appear to be the best approach. In either case, after filling the grooves the filling material is cured at room temperature, though somewhat warmed by laying a heated aluminum block on top, separated with a Teflon sheet. The option to put this in an oven to cure is not consistent with maintaining exact repeatability of position. Rather the filling and curing is done without moving the card sandwich from the cutting tool or allowing it to shift in any way. It is also necessary to drain the water flooding the cutting area, rinsing with distilled water, and drying the grooves thoroughly.

Now the second cut can be made as shown in Part (d). The cut out part 77 leaves a small part of transducer material 1 as shown in the cross section. This is the necessary thin strip that runs the width of the card. This second cut is done with the structural support benefit of the supporting material 72 that was filled into the groove on the other side of the strip. The supporting material and the upper foundation material provide interlocking support of the transducer material and the attached metal surfaces. These metal surfaces are too thin to show in this view. The result of this second cut is the final transducer material part 1 and the upper foundation material strip 4. This second cut type is then repeated, as shown in this Part (d) diagram.

Part (e) shows how the remaining cuts trim to form the strip of supporting material 2 and the lower foundation material strip with a flange 78. They also trim the top, which has an uneven quality after filling and an excess thickness of upper foundation material, both not shown. Then the assembly is completed by a separating cut 79.

End trimming must also be done. It is a separate, and final, set of operations that do not have such critical precision issues. It is done after moving the hold down. clamps to expose the edges, with the new hold down position being in the middle of the sandwich. Clamping is now over delicate strip parts but they are very flat so there is not a breakage problem. The process of cutting the transducer material end tabs where there is metal on the tab surfaces has been a troublesome part of the manufacturing process. For very thin tabs of transducer material, there is insufficient strength to enable any kind of cutting, and the metal surfaces greatly aggravate this by tending to gum up the cutter, and peel back rather than cut cleanly. This led to the end enclosure design that keeps the tabs enclosed and supported during final cutting, including the final cutting that separates the multiple assemblies. The material formulation used for the foundation material cards was used successfully. The actual cutting operations are self evident from the illustration of that end part 7 shown in FIG. 1. In actual practice, this material has been inserted and cured at the same time as the card sandwich was assembled, bonded, and cured. A mold tool was used that clamped the cards together while leaving openings at the edges for the end enclosure material to be inserted, and then side strips were inserted to contain the end enclosure material while it cured. Wires were added after the assemblies were separated from each other. This required grinding operations with a hand held tool to expose the metal surfaces on the tabs.

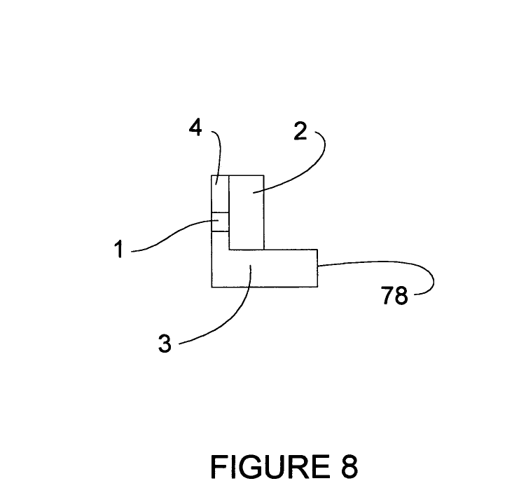

FIG. 8 shows a cross section of a final, single assembly that results from the previously discussed process. Here a piezo-electric transducer material strip 1, a backing material strip 2, a lower foundation material strip 3 with a flange 78, and an upper foundation material strip 4 are shown.

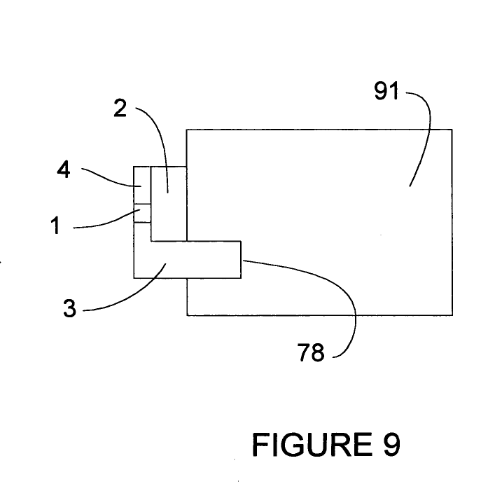

FIG. 9 shows the same part mounted with the flange 78 used to position the transducer assembly on a backing block 91. This is representative of a typical transducer system where many of such strips are mounted in grooves on that same backing block.

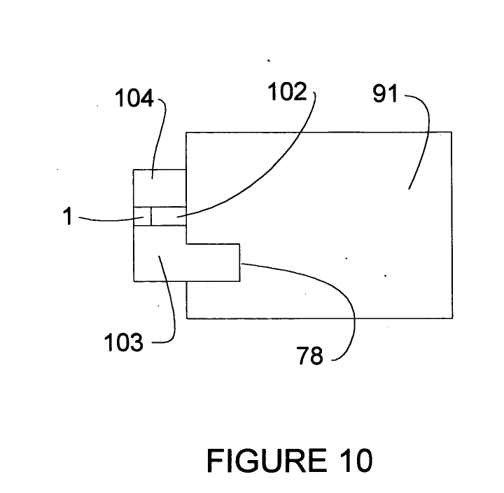

FIG. 10 shows a similar transducer assembly made with a modified process that does not use epoxy as the supporting material filler. Here the strip of supporting material is a continuation of the same ceramic as the transducer material. In this form the transducer part is made separate from an inoperative part of the ceramic, where the inoperative part is caused by removal or isolating the conductive surface part that interacts with electric field effects in the ceramic. This means that the coupling of ultrasonic waves with the active transducer material part with the supporting material part will be very effective. FIG. 10 shows this variation mounted on a backing block 91. A backing block may be a stack of thin backing blocks. In many circumstances, the impedance into the material of the backing block must be addressed in order to get the desired transducer system response. In the initially described arrangement, the impedance of the supporting material may be an issue for some applications, where there would need to be variations in the composition of the filling material parts. Impedance effects and impedance matching layer effects are known topics in the field of ultrasonics.

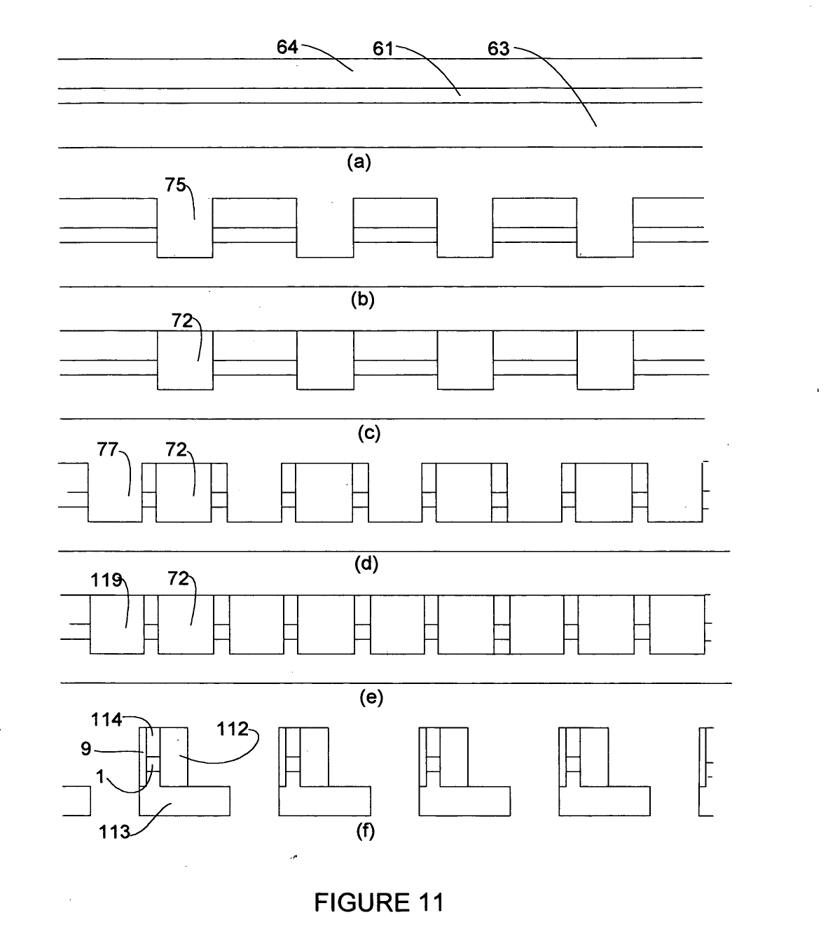

A variation on the design shown in FIGS. 7,8,9 is to add a strip of supporting material to interface with the intended medium of ultrasonic wave propagation. This is illustrated in FIG. 11. This can be done in addition to the previously discussed supporting strip, or it can be done instead of that strip. FIG. 11 shows a modified process using an additional filling operation that includes inserting a front matching layer material 119 that is inserted in the step indicated by Part (d). Trimming is then done to provide a quarter wavelength layer 9 of a matching layer material selected to form an impedance matching system to enhance coupling of ultrasonic wave energy into a medium of operation. In this example the lower and upper foundation strips 113,114 are slightly different, as is the rear supporting material strip 112.

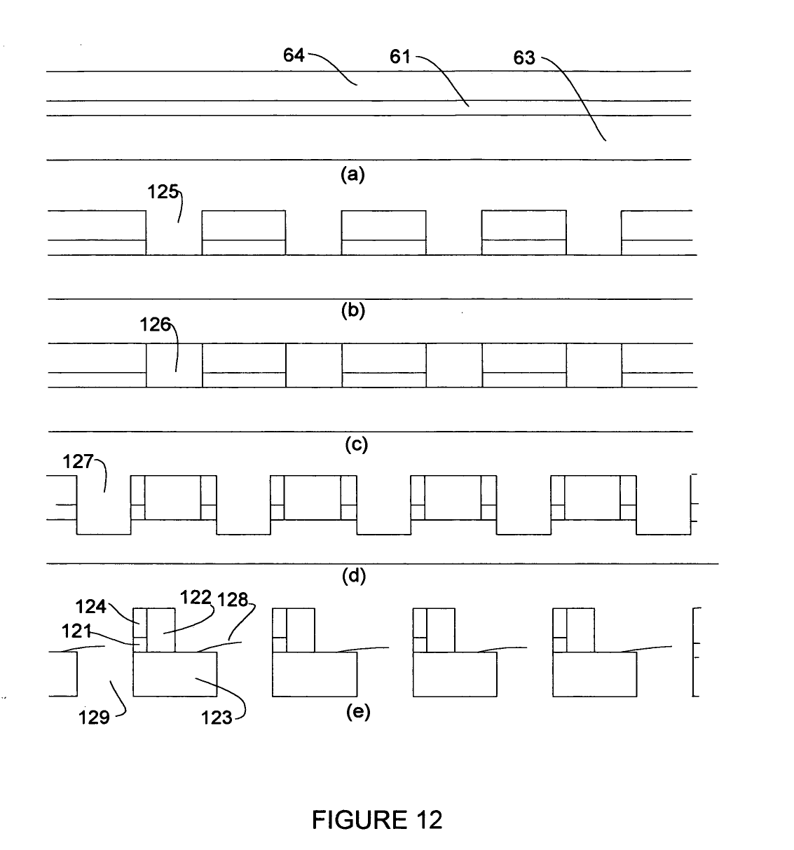

FIG. 12 shows variations necessary to segment the transducer assembly strip into a number of independent elements. Part (a) appears to be show the same card sandwich as before, except the card here represented has been previously cut with longitudinal grooves that are parallel to the plane of the cross section that is shown. In the orientation shown, these longitudinal grooves were made by cuts from the lower side of the card sandwich. They penetrate upward through the lower layer of foundation material, the lower conductive surface, and most of the transducer material. Such longitudinal cuts stop before disturbing the upper conductive surface or the upper layer of foundation material. The longitudinal grooves are filled with a material similar to that of the foundation material as previously specified to isolate individual elements. Transverse grooves 125 of FIG. 12, Part (B) are orthogonal to the longitudinal grooves. The transverse grooves are cut as before, but these grooves are not as deep so as to not break the lower conductive surface. After filling these 126 as before, a precise cut 127 is made to fix the width of the transducer material strip 121 that is now segmented due to the longitudinal cuts. The flange of foundation material 123 is as before but it is also segmented. FIG. 12, Part (e) shows the result after trimming to form supporting strip 122 and making cut off cut 129. Also, individual wires 128 are added that connect to the corresponding segmented transducer elements.

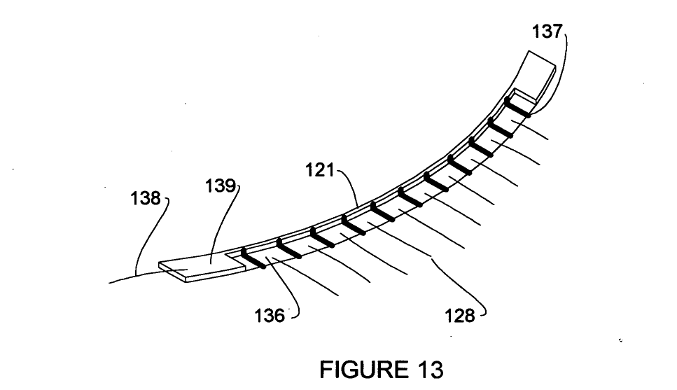

A single segmented strip transducer assembly is illustrated with an isometric view in FIG. 13. A segment 121 is shown in a long strip of such segments. The long strip is overlaid with a continuous conductive surface 139 that is tied to the end tab surfaces where a wire 138 is attached. The opposite side conductive surface, now segmented, is now accessible through conductive tabs 136. Wires 128 are attached to the conductive tabs, and the wires are fed through the backing block to enable connection to electronic devices. The strip of segments is shown laid out along a curved line, which is only representative of the ultimate line shapes that are anticipated for general two dimensional aperture array systems that this segmented variation enables.

This description of the preferred embodiments has provided illustrations of the invented device and methods for its manufacturer. As such it demonstrates a concept that is expected to have many variations that are a result of this invention. The appended claims should determine the scope of this invention, rather than the examples given.

I claim:

1. A transducer assembly that includes a first thin strip of transducer material, where said transducer material operates to convert between electrical and ultrasonic forms of energy, where said first thin strip has conductive surfaces attached to a first side and an opposite second side, and a second thin strip of reinforcing material, where said second thin strip is attached to a third side of said first thin strip, and where said reinforcing material has ultrasonic properties that support ultrasonic wave propagation through said second thin strip.

2. A transducer assembly according to claim 1 where said supporting material was arranged against said third side of said first thin strip to provide structural support to said transducer material during a cutting operation that was used in forming said first thin strip.

3. A transducer assembly according to claim 1 and a backing block, where said supporting material has ultrasonic properties that enable ultrasonic wave propagation between said second thin strip and said backing block.

4. A transducer assembly according to claim 1 and a backing block, where said supporting material has characteristic ultrasonic impedance that matches that of said backing block.

5. A transducer assembly according to claim 1 and a backing block, where said supporting material is formed in a controlled shape to enable a controlled position of said thin strip of transducer material relative to said backing block.

6. A transducer assembly according to claim 1 and a backing block, where said supporting material is formed to have a flange that aids in assembly of said transducer assembly with a backing block, such that said transducer assembly is accurately positioned.

7. A transducer assembly according to claim 1 adapted for operation in a medium of wave propagation, where said supporting material has ultrasonic properties that enable ultrasonic wave propagation through said second thin strip between said medium of wave propagation and said first thin strip.

8. A transducer assembly according to claim 1 adapted for operation in a medium of wave propagation, where said supporting material has ultrasonic properties that enable ultrasonic wave propagation through said second thin strip between said medium of wave propagation and said first thin strip, such that second thin strip provides an impedance matching layer function.

9. A transducer assembly according to claim 1 where at least one of said conductive surfaces is an electroplated surface that provides a controlled conductive path in said transducer assembly.

10. A transducer assembly according to claim 1 where, for a side having a conductive surface attached thereto, there is also a thin strip of holding material attached thereto, where said holding material has ultrasonic properties that suppress propagation of ultrasonic waves through said thin strip of holding material.

11. A transducer assembly according to claim 1 where, for a side having a conductive surface attached thereto, there is also a thin strip of holding material attached thereto, where said holding material has structural properties that contributed to holding said transducer material in position during cutting operations.

12. A transducer assembly according to claim 1 where, for a side having a conductive surface attached thereto, there is also a thin strip of holding material attached thereto, and a backing block, where said holding material extends to provide a flange that enables accurate assembly of said transducer assembly with said backing block.

13. A transducer according to claim 1 where said first thin strip is segmented to form a plurality of independent transducer elements.

14. A transducer according to claim I where said first thin strip is segmented and a conductive surface is segmented to form a plurality of conductive surface patches that are attached to respective said plurality of independent transducer elements.

15. A transducer according to claim 1 where said second thin strip of reinforcing material is of material that is identical to said transducer material except that it is rendered inactive as transducer material by control of conductive surface placement.

16. A method of manufacturing a transducer device where said transducer device includes a thin strip of transducer material, said method including steps of
(a) cutting a first groove in a card of transducer material to cause a first edge,
(b) inserting supporting material in said first groove such that said supporting material is conformal and attached to said first edge,
(c) cutting a second groove in said card of transducer material to cause a second edge, where said first edge and said second edge bound said thin strip of transducer material to establish a controlled width of said thin strip of transducer material, where said supporting material contributes to stability of said thin strip of transducer material during operation of said cutting a second groove.

17. A method according to claim 16 where said supporting material is formed into a thin strip of supporting material that remains a part of said transducer device, where said thin strip of supporting material has ultrasonic properties that support ultrasonic wave propagation in relation to said strip of transducer material.

18. A method according to claim 16 where said supporting material is inserted into said first groove in the form of a liquid, where said liquid cures to form a solid.

19. A method according to claim 16 where said supporting material is inserted into said first groove in the form of a combination of a solid and a liquid, where said combination of a solid and a liquid cures to form a solid, where distortion of said first groove as a result of curing is minimized.

20. A method according to claim 16 where said supporting material is formed into a thin strip of supporting material that remains a part of said transducer device, where said thin strip of supporting material is trimmed to form a flange that aids in accurate positioning of said transducer device.

21. A method according to claim 16 with a preliminary step of attaching a card of foundation material to said card of transducer material, where said card of foundation material improves stability of said thin strip of transducer material during cutting operations.

22. A method according to claim 16 with a preliminary step of attaching a card of foundation material to said card of transducer material, where subsequent cutting steps cause said card of foundation material to be formed into a strip of foundation material, where said strip of foundation material remains a part of said transducer device

23. A method according to claim 16 with a preliminary step of attaching a card of foundation material to said card of transducer material, where subsequent cutting steps cause said card of foundation material to be formed into a strip of foundation material, where said strip of foundation material remains a part of said transducer device, where ultrasonic characteristics of said foundation material are such that ultrasonic wave propagation through said foundation material is suppressed.

24. A method according to claim 16 with a preliminary step of attaching a card of foundation material to said card of transducer material, where subsequent cutting steps cause said card of foundation material to be formed into a strip of foundation material, where said strip of foundation material remains a part of said transducer device, where ultrasonic characteristics of said foundation material are such that ultrasonic wave propagation through said foundation material is suppressed and constraint of displacement of a side of said strip of transducer material is minimized.

25. A method according to claim 16 with a preliminary step of attaching a card of foundation material to said card of transducer material, where subsequent cutting steps cause said card of foundation material to be formed into a strip of foundation material, where said strip of foundation material remains a part of said transducer device, and where said strip of foundation material is formed such that a flange is provided that aids in positioning of said transducer device.

26. A method according to claim 16 with a preliminary step of attaching two cards of foundation material to respective sides of said card of transducer material, where said two cards of foundation material improve stability of said thin strip of transducer material during cutting operations.

27. A method of manufacturing a transducer device that includes a plurality of independent transducer elements, where said transducer device includes a thin strip of transducer material that is segmented, said method including steps of
(a) cutting longitudinal grooves in a card of transducer material, where said card of transducer material has a back side and a front side with conductive surfaces attached to both sides, where said longitudinal grooves are cut from said back side through most of said card of transducer material to a controlled depth that prevents cutting of a front side conductive surface,
(b) cutting a first transverse groove in a card of transducer material to cause a first edge, where said transverse groove is cut from said front side through most of said card of transducer material to a controlled depth that prevents cutting of a back side conductive surface,
(c) inserting supporting material in said first transverse groove such that said supporting material is conformal and attached to said first edge,
(d) cutting a second transverse groove in said card of transducer material to cause a second edge, where said first edge and said second edge bound said thin strip of transducer material to establish a controlled width of said thin strip of transducer material, where said supporting material contributes to stability of said thin strip of transducer material during operation of said cutting a second groove, and
(e) cutting to expose back side conductive surfaces that are associated with respective transducer elements such that electrical contact can be made with said back side conductive surfaces.

28. A method according to claim 27 where said longitudinal grooves are filled with material that contributes to stability of said transducer material during cutting operations.

29. A method according to claim 27 where said longitudinal grooves are filled with material having ultrasonic properties that supports independent operational characteristics of said transducer element.

30. A method according to claim 27 where an electrical connection is made to a tab that is a remnant part of said front side conductive surface that remains with said transducer device.
